(19) United States
(12) Patent Application Publication
(10) Pub. No.: US 2026/0191524 A1
(43) Pub. Date: Jul. 9, 2026

(54) SURGICAL SUTURING INSTRUMENT

(71) Applicant: Yihe LI, BEIJING (CN)

(72) Inventors: Yihe LI, BEIJING (CN); Yanfeng LI, BEIJING (CN); Yijie LIANG, BEIJING (CN)

(21) Appl. No.: 19/295,460

(22) Filed: Aug. 8, 2025

(30) Foreign Application Priority Data
Jan. 9, 2025 (CN) 202510036732.4

(51) Int. Cl. A61B 17/04; A61B 17/00; A61B 17/06

(52) U.S. Cl. CPC A61B 17/0482; A61B 17/06066; A61B 17/06166; A61B 2017/00004; A61B 2017/00938; A61B 2017/00942; A61B 2017/06185

(57) ABSTRACT

The present application refers to the technical field of medical device and specifically relates to a surgical suturing instrument for reducing fluid accumulation around suture site. The surgical suturing instrument comprises a suture needle and a suture thread, connected with each other. The present application can perform liquid drainage of sutured area, avoiding various complications such as edema, delayed healing, infection, etc. which are caused by ineffective drainage of blood or tissue fluid in the wound area. In addition, the drainage channels can ensure that during the suturing operation, the negative pressure restriction of tissue surrounding the suture and needle can be lifted.

### CROSS REFERENCE TO RELATED APPLICATION

This application claims priority to Chinese patent application No. 202510036732.4, filed on Jan. 9, 2025, entitled ‘Suture with drainage function and surgical suturing instruments using the suture.

### TECHNICAL FIELD

The present invention belongs to the field of medical device technology, and specifically relates to a surgical suturing instrument.

### BACKGROUND

Surgical suturing is a crucial step in the surgical process, primarily aiming at restoring tissue integrity and promoting wound healing. However, traditional suturing techniques often face the problem of poor drainage in the surgical area after surgery, leading to the accumulation of blood and tissue fluids in the surgical area, causing tissue swelling, increasing the risk of infection, and possibly prolonging the patient's recovery time.

Although various technologies and instruments have been developed in the past few decades to enhance suture effectiveness and accelerate patient recovery, including the use of staples, skin glue, and suture needles and threads of different shapes and sizes made of various materials, these improvements provide more choices for various types of wounds. However, existing technology still cannot completely avoid the accumulation of blood and tissue fluid in the suture area, which often leads to edema and other complications.

Therefore, there is an urgent need for a suturing instrument which is simple, easy to use and has drainage function to better address the limitations of existing suture techniques, reducing the risk of complications, and improving postoperative recovery efficiency.

### CONTENTS OF THE INVENTION

The present invention proposes a surgical suturing instrument for reducing fluid accumulation around suture site, wherein the surgical suturing instrument comprises a suture needle and a suture thread, so as to address the shortcomings of existing technology. By setting a drainage channel on the suture thread, efficient drainage of tissue fluid in the suture area is achieved, thereby achieving the goal of synchronous suturing and drainage, effectively improving postoperative wound management and healing effects.

On one aspect, the present invention provides a surgical suturing instrument for reducing fluid accumulation around suture site, wherein the surgical suturing instrument comprises a suture needle and a suture thread, connected with each other; at least one guide groove is provided on the suture needle, wherein the suture thread comprises a suture body; a drainage channel is provided in the middle or at a side of the suture body, which extends along its length direction; the drainage channel is connected to a tissue needle channel which is caused by a needle that passing through the tissue during suturing process, and the drainage channel is used to drain fluid that seeped out of the tissue needle channel and suture area.

By adopting the above technical solution, the drainage channel set in the middle or at side of the suture body can penetrate through the suture body along the length direction of the suture, ensuring that the leaked liquid can be quickly drained away. This design effectively reduces the accumulation of blood and tissue fluid in the sutured area after surgery, lowers the risk of tissue swelling, and helps to reduce infections. By timely drainage of exudate, the retention and pressure of fluid at the edge of the wound can be reduced, thereby reducing the likelihood of inflammatory reactions and scar hyperplasia caused by fluid accumulation. This surgical suturing instrument is suitable for various surgical sutures, especially for areas with high osmotic fluid or high risk of infection, such as abdominal and chest surgeries.

Optionally, in the surgical suturing instrument, the structure of the suture thread adopts a single strand thread body or a multi strand thread body;
The suture thread of the single stranded suture body is provided with an opening on its side that is connected to the drainage channel and the tissue needle channel, which opening is used to drain the liquid in the tissue needle channel and the suture area to the drainage channel; the multi-strand thread bodies are connected to each other through a connecting part, and the connecting part is set along the suture thread path; wherein the connecting part is concave inward relative to the surface of the wire body connected to it, and the concave is used as a drainage channel; the number of multi strand thread bodies is 2-6.

By adopting the above technical solution, the single-strand suture body is equipped with an opening on the side that is connected to the drainage channel and tissue needle channel. Such design can effectively guide the exudate in the suture area and tissue needle channel to enter the drainage channel. The multi-strand suture body is connected by the connecting part, so that each strand of suture body maintains a stable structure. The depression formed by the connecting part creates a drainage path between different suture bodies, which is suitable for dealing with complex situations of liquid leakage in the surgical area and enhances the adaptability of the suture thread. The design of multi-strand suture provides a flexible choice of 2-6 strands, and medical staff can choose an appropriate number of strands of suture thread according to specific surgical needs to adapt to different wound sizes and shapes, thereby achieving personalized suture plans.

Optionally, there are multiple holes arranged along the side of the suture thread, or a side groove extending along the longitudinal axis of the suture thread and penetrating its entire length.

When the opening of the suture thread of the single strand thread body adopts a side groove, the opening angle of the edges on both sides of the cross-section of the suture thread is greater than 0° and less than 180°.

By adopting the above technical solution, the design of the opening can adopt multiple holes arranged along the side of the suture thread, or a side groove extending along the longitudinal axis of the suture thread and running through the entire length. This diversified drainage path ensures that exudate can be guided to the drainage channel under different surgical conditions.

When the opening is a side groove with an angle greater than 30° and less than 90°, this angle range can provide sufficient drainage space to prevent the drainage channel from closing due to tissue compression.

Optionally, the drainage channel extends along the longitudinal axis of the suture.

By adopting the above technical solution, the drainage channel is designed to extend along the longitudinal axis of the suture thread, so that the drainage effect can cover the entire length of the suture thread, ensuring the perforation and continuity of the drainage channel, and enabling effective drainage of all parts of the wound.

Optionally, the opening edge of the suture with a single strand suture body has a suture flank extending outward, which extends from one end of the suture body to the other along its longitudinal axis.

By adopting the above technical solution, side wings of the suture can stretch open the soft tissue around the suture body, preventing the drainage channel from being compressed and closed by surrounding soft tissue.

Optionally, the cross-sectional shape of the drainage channel with single strand suture body adopts one of below shapes: crescent shape, semi-circular shape, square shape, and elliptical shape.

By adopting the above technical solution, the cross-sectional shape of the drainage channel adopts diverse shapes, which can optimize the drainage performance according to different surgical needs.

Optionally, the material of the suture thread may be non degradable materials and/or biodegradable materials; Non degradable materials include one or more of below material: nylon, polytetrafluoroethylene, polyester fiber, stainless steel, nickel chromium alloy, titanium, and titanium alloys; Biodegradable materials include one or more of below material: polyacetic acid, polylactic acid hydroxyacetic acid copolymer, polyhydroxyacetic acid ethyl ester, magnesium and magnesium alloys, iron and iron alloys, zinc and zinc alloys.

By adopting the above technical solution, the suture thread is designed with non degradable and/or biodegradable materials, providing a wide range of material choices. Non degradable materials have excellent strength, durability, and biocompatibility, making them suitable for surgeries that require long-term suture support, such as joint repair and heart valve fixation; Biodegradable materials can gradually degrade in the body, making them suitable for situations that do not require permanent suture support, such as skin suturing, muscle repair, and visceral surgery, which can help reduce postoperative foreign body reactions and the need for secondary surgery. The flexible selection of materials enables sutures to adapt to a wide range of clinical scenarios and provide precise postoperative support.

Optionally, the inner and outer surfaces of the suture are coated with a hydrophobic coating containing anti infective drugs; Or the inner and outer surfaces of the suture are coated with a hydrophilic coating containing anti infective drugs.

By adopting the above technical solution, the inner and outer surfaces of the suture are coated with a coating containing anti-infective drugs. Whether it is a hydrophobic coating or a hydrophilic coating, it can effectively release anti-infective drugs, directly acting on the suture area, inhibiting bacterial growth and reproduction, reducing the risk of postoperative infection, improving surgical safety and patient recovery.

The surgical suturing instrument can use any of the sutures described herein.

Medical staff can choose suitable sutures according to the type of surgery and the specific situation of the patient, achieving personalized surgical plans and meeting different clinical needs. The guide groove set on the suture needle can effectively relieve the negative pressure restriction of surrounding tissues on the suture needle during dynamic threading, reducing the adsorption and friction of tissues on the needle body.

Optionally, the guide groove is longitudinally arranged on the surface of the needle body of the suture needle, starting from the part of the needle body near the needle tip, smoothly extending along the curved surface of the suture needle until the needle tail.

By adopting the above technical solution and setting a smoothly extending guide groove, the pressure exerted by the suture needle on the tissue during the puncture process is effectively dispersed, thereby reducing the cutting and compression damage to the tissue. This design helps to reduce intraoperative bleeding and tissue trauma, promote postoperative recovery, and reduce the occurrence of complications.

In summary, the present invention includes at least one beneficial technical effect as follows:
Firstly, the drainage channel and channel design of the present invention can guide the exudate of the surgical area, reduce the accumulation of blood and tissue fluid, lower the risk of postoperative infection and tissue swelling, and promote wound healing.
Secondly, the multi-strand suture design of the present invention provides flexible selection of 2-6 strands, and doctor can select appropriate number of suture bodies according to surgical needs to adapt to different wound sizes and shapes, providing personalized suture solutions.
Thirdly, the suture thread of the surgical suturing instrument is designed with side opening of a groove shape and suture side wings to prevent the drainage channel from closing due to soft tissue compression, ensuring the perforation and continuity of the drainage channel.
Fourthly, the suture thread of the surgical suturing instrument can use non degradable or biodegradable materials to meet the requirements of different surgeries for durability and biocompatibility. Non degradable materials provide long-term support, while biodegradable materials reduce foreign body reactions and secondary surgical needs.
Fifthly, the suture thread of the surgical suturing instrument can be coated with a hydrophobic or hydrophilic coating containing anti-infective and anticoagulant drugs, effectively preventing infection and improving the lubricity of the suture operation. Hydrophobic coatings reduce liquid adhesion, while hydrophilic coatings enhance operational performance, meeting different postoperative care needs.
Sixth, by setting up a guide groove, the negative pressure restriction of the tissue on the suture needle is reduced, improving the efficiency and accuracy of suturing.

### DRAWINGS DESCRIPTION

FIG. 1 is a schematic diagram of the structure of the suturing instrument of the present invention;

FIG. 2 is a three-dimensional structural diagram of the suture needle of the present invention;

FIG. 3 is a physical photograph of the suturing instrument of the present invention.

FIG. 4 is a three-dimensional structural diagram of a single stranded suture thread according to embodiment 1 of the present invention;

FIG. 5 is a side view of the single stranded suture thread of Embodiment 1 of the present invention;

FIG. 6 is a partial photograph of the single stranded suture thread in embodiment 1 of the present invention;

FIG. 7 is a partial photograph of the single stranded suture thread in embodiment 1 of the present invention;

FIG. 8 shows the effect of the existing single stranded suture without drainage channel in the suture area;

FIG. 9 shows the effect of a single suture thread in the suture area of Example 1 of the present invention;

FIG. 10 is a three-dimensional structural diagram of a single stranded suture thread with a structure different from FIG. 1 of the present invention;

FIG. 11 is a three-dimensional structural diagram of the double stranded suture thread in embodiment 2 of the present invention;

FIG. 12 is a physical photograph of the double stranded suture thread in embodiment 2 of the present invention;

FIG. 13 shows the effect of the double stranded suture in the suture area of embodiment 2 of the present invention;

FIG. 14 is a three-dimensional structural diagram of the three strand suture thread in embodiment 3 of the present invention;

FIG. 15 is a three-dimensional structural diagram of a single stranded suture thread with a variant structure according to embodiment 3 of the present invention;

FIG. 16 is another schematic diagram of the suturing instrument of the present invention.

wherein, 1—suture thread; 10—Drainage channel; 11—Holes; 12—Connection part; 13 Suture flank; 2—Transition section; 3—Suture needle; 30—Diversion groove.

### EMBODIMENTS

In order to enable the skilled in the art to better understand the technical solution of the present application, the following will provide further detailed explanations of the present application in conjunction with the accompanying drawings and embodiments.

In the description of this application, the terms “first”, “second”, and “third” are used for descriptive purposes only and should not be understood as indicating or implying relative importance; The term ‘multiple’ refers to two or more, unless otherwise specified. The terms ‘installation’, ‘connection’, ‘connection’, ‘fixation’, etc. should be broadly understood. For example, ‘connection’ can be a fixed connection, a detachable connection, or an integral connection; ‘Connected’ can be directly connected or indirectly connected through an intermediate medium. For ordinary skilled in the art, the specific meanings of the above terms in this application can be understood according to the specific situation.

In the description of this application, it should be understood that the terms “up”, “down”, “left”, “right”, “front”, “back”, etc. indicating orientation or positional relationships are based on the orientation or positional relationships shown in the accompanying drawings. This is only for the convenience of describing this application and simplifying the description, and does not indicate or imply that the device or unit referred to must have a specific orientation, be constructed and operated in a specific orientation.

Therefore, it cannot be understood as a limitation of this application.

The present invention describes a suture thread with drainage function and a surgical suturing instrument using the suture thread. There is a drainage channel that runs along the suture thread in the longitudinal direction, and the suture thread is connected to the suture needle for use. When the suture needle penetrates into skin or muscle tissue, a needle channel is formed, and the suture thread follows the needle body into the needle channel. The drainage channel timely exports exudate around the tissue needle channel, effectively avoiding blood or tissue fluid retention, reducing the occurrence of wound complications, and promoting wound healing.

### Example 1

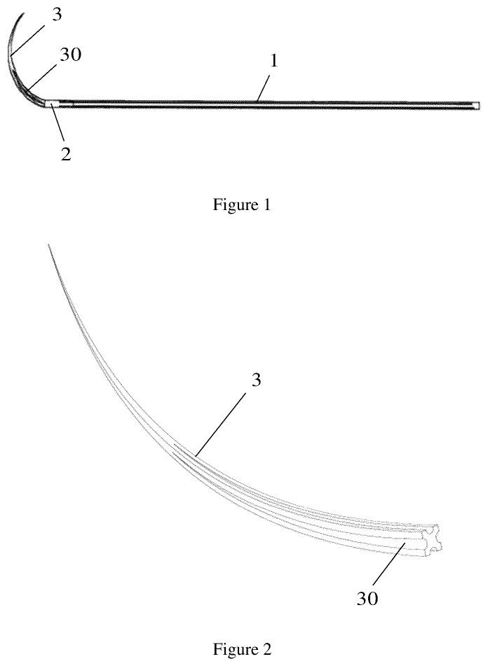

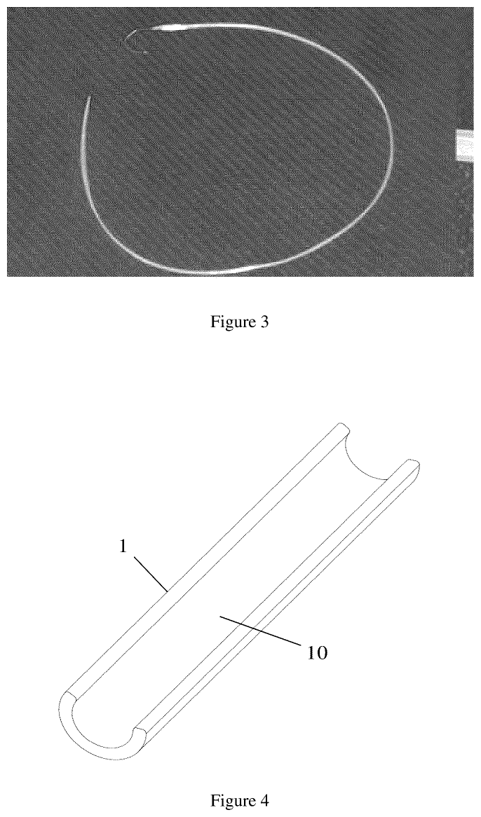

As shown in FIGS. 1-3, the present invention provides a surgical suturing instrument, which includes a suture needle 3 connected to the suture thread, and a transition portion 2 connecting the suture thread and the suture needle 3.

The surface of suture needle 3 has one or more longitudinally extending guide grooves 30, which start near the needle tip and extend all the way to the end of the needle. The diversion groove 30 is used to help discharge excess fluids, such as oozing blood, during the suturing process, preventing these fluids from staying at the surgical site. This can maintain a clear surgical field of view and avoid affecting the surgical process. These guide grooves 30 can also reduce the negative pressure of the tissue around the suture on the suture needle, making the threading process smoother.

The suture can be any of the suture with drainage function described below. The use of sutures with drainage function helps to drain exudate during surgery and reduce postoperative complications.

The transition part connecting the suture needle and suture thread is designed to gradually become thinner from the end of the suture needle to the end of the suture thread. This design can enhance the stability of the connection, reduce sudden transitions between the suture and the needle body, and prevent blood or tissue fluid leakage caused by mismatched connection sites. The diameter of the transition section is slightly larger than the suture thread, which allows the suture thread to smoothly enter the surgical area through the transition section.

Optionally, the cross-section of suture needle 3 can be circular, triangular, elliptical, or shovel shaped, which helps the needle holder to grip suture needle 3 more firmly during surgery and prevent it from slipping or deviating. According to the needs, the suture needle can also be straight or curved, with a curvature of ¼ arc, ⅜ arc, ½ arc, ⅝ arc, etc., to meet different surgical requirements.

Optionally, according to the cross-section of the suture needle 3, the guide groove 30 can be arranged at single-side, at double-sides, at three sides, arranged circumferential, or distributed at multi-stages, or arranged in a combination manner of the above arrangements. Single-side arrangement means that the flow channel is located on one side of the suture needle, extending from near the needle tip to the needle tail. Single-sided arrangement can effectively guide the discharge of fluid from the surgical site, suitable for surgical scenarios that require a small drainage volume. Bilateral symmetrical arrangement (arranged at double-sides) refers to two guide grooves symmetrically arranged on both sides of the suture needle, extending along the curved surface of the suture needle. This arrangement can increase drainage efficiency and is suitable for surgical scenes with a lot of exudate. It can provide a more balanced drainage effect and effectively avoid the deviation and unevenness that may be caused by unilateral drainage. Three-sided arrangement refers to setting drainage grooves on the three surfaces of the suture needle to form a multi-faceted drainage design, ensuring that the liquid can be smoothly drained during the surgical process regardless of changes in the rotation angle of the suture needle. Surrounding arrangement means that the guide groove is arranged in a spiral shape on the surface of the suture needle, extending from the needle tip to the needle tail. This design can provide a continuous drainage path during the needle insertion process, without direction restrictions. Multi segment distributed arrangement means that the guide grooves are arranged in multiple segments on the surface of the suture needle, forming spaced drainage paths. There is a certain interval between each guide groove to reduce the weakening to the needle body strength. It is also possible to combine the various arrangement forms mentioned above, and according to the surgical needs and the design characteristics of the suture needle, arranging the guide groove as a complex shape that meets specific surgical requirements.

### Detailed Description of the Suture Thread

As shown in FIG. 4, the suture thread with drainage function of the present invention includes a suture thread body, and a drainage channel 10 provided in the middle or side of the suture thread body along its length direction. The drainage channel 10 is used to drain the liquid (such as blood or tissue fluid) leaked during the surgical process to the outside of the suture area, preventing the accumulation of liquid and causing complications.

Suture 1 is composed of a single stranded thread body, and an opening is provided on its side to connect with drainage channel 10 and tissue needle channel. The function of the opening is to allowing the liquid in the needle passage and suture area to enter the drainage channel 10, ensuring smooth drainage of the liquid and reducing the risk of postoperative infection and swelling. The opening is a side groove that runs along the longitudinal axis of suture thread 1.

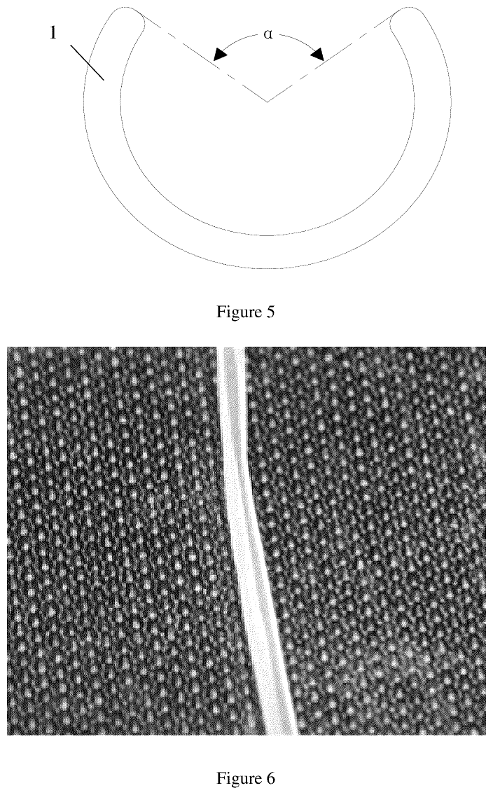

As shown in FIG. 5, when the suture is in a straight state and under no force, the opening angle α of the edges on both sides of the cross-section of suture 1 is between 0° and 180°. A angle (close to 0°) too small may make the channel narrower and more susceptible to external pressure. A larger angle (close to 180°) will increase the openness of the drainage channel, but it may affect the overall strength and operability of the suture. In order to optimize the drainage effect and the structural stability of the suture, the preferred range of angle α is 30°to 90°. Choosing an angle between 30° and 90° can not only optimize the smoothness of liquid drainage and reduce the risk of channel closure caused by tissue compression, but also ensure that the suture maintains high mechanical strength and stability during operation.

The cross-section of the drainage channel can adopt various shapes, such as crescent shaped, semi-circular, square, or elliptical, depending on the surgical needs. This design helps to maintain the smoothness of the channel and effective drainage effect.

In this embodiment, the wall thickness of suture thread 1 is 0.2~0.5 times the diameter of drainage channel 10. If the wall thickness is set too small, it will cause the suture to break due to excessive tension during the suturing process; if the thickness is set too large, it may affect the flexibility of the suture and the effectiveness of the drainage channel. Choosing a ratio of 0.2 to 0.5 effectively avoids these two types of problems, ensuring a balance between drainage function and structural integrity. The recommended range is 0.3 to 0.4 times the diameter of drainage channel 10. Within this range, the suture can provide sufficient tensile strength and avoid the risk of fracture caused by a thin wall thickness. The design of wall thickness directly affects the ultimate tensile strength of the suture. By controlling the ratio of wall thickness to drainage channel diameter between 0.2 and 0.5, it is ensured that the suture can withstand effective tension that required for wound closure during use. In surgeries that require higher intensity, such as joint repair and abdominal incision surgery, a wall thickness close to 0.5 times the ratio can be chosen to enhance tensile strength. In soft tissue or surface suturing surgeries, a wall thickness close to 0.2 times the ratio helps increase flexibility and ease of operation.

Optionally, the material of suture thread 1 adopts non degradable materials and/or biodegradable materials. Non degradable materials include one or more of nylon, polytetrafluoroethylene, polyester fiber, stainless steel, nickel chromium alloy, titanium, and titanium alloys; Biodegradable materials include one or more of polyacetic acid, polylactic acid hydroxyacetic acid copolymer, polyhydroxyacetic acid ethyl ester, magnesium and magnesium alloys, iron and iron alloys, zinc and zinc alloys. These materials all have a certain mechanical strength to ensure that suture thread 1 will not induce compression deformation of drainage channel 10 due to expansion by absorption of blood or tissue fluid or suture distortion during use, ensuring smooth drainage during the suture process. In clinical, material combinations can be selected based on the type of surgery (such as orthopedics, plastic surgery, gastrointestinal surgery) to meet the specific mechanical, drainage, and biocompatibility requirements of the surgery. For example, for soft tissue repair surgery (such as skin suturing and plastic surgery), the suture thread needs to have good flexibility and biocompatibility, and the drainage channel should be able to prevent postoperative exudate accumulation. Nylon and polytetrafluoroethylene materials are adopted, since they possess soft and low irritation characteristics, suitable for soft tissue suturing without affecting the natural healing process of the tissue. The drainage channel helps to keep the incision area dry, reduce infection and scar formation. For surgeries in high-risk areas such as the abdomen and urogenital system, these surgical sites require high infection control and may require temporary drainage function. For such surgeries, by adopting biodegradable polymer materials, such as polylactic acid hydroxyacetic acid copolymers, it can provide temporary support and gradually degrade, reducing foreign body reactions. Titanium alloy is suitable for scenarios that require high-strength support, and the anti infection coating on its surface can significantly reduce the risk of infection. For orthopedic surgeries such as joint repair and fracture fixation, extremely high tensile strength and durability are required, while adapting to the biomechanical environment of the bone. For such surgeries, stainless steel and nickel chromium alloy are adopted, to provide excellent mechanical strength and biocompatibility, suitable for long-term retention. Magnesium alloys have a fast degradation rate and are suitable for situations where long-term support is not required after bone healing. For cardiovascular surgery (such as vascular suturing and valve repair), sutures need to have good blood compatibility, low friction, and tensile strength to prevent thrombosis and tissue reactions. For such surgery, polytetrafluoroethylene and polyester fibers are adopted, which have good flexibility and biocompatibility.

Optionally, the inner and outer surfaces of suture 1 are coated with a hydrophobic coating containing anti infective and anticoagulant drugs; Or coating with hydrophilic coatings containing anti infective and anticoagulant drugs. Hydrophobic coatings can repel moisture and body fluids, reduce the adhesion of liquids on the surface of sutures, and lower friction and adhesion between tissues and sutures. Suitable for surgeries that require reduced fluid contact and adhesion, such as suturing in the abdominal cavity, chest cavity, and joints. The hydrophilic coating absorbs moisture, increases the lubricity of the suture, making it smoother during puncture and suturing, and reducing tissue damage. Hydrophilic coatings are suitable for suturing operations that require high smoothness, such as vascular suturing, cardiovascular surgery, soft tissue repair, etc.

This embodiment takes mold extrusion molding and laser cutting technology as examples to explain the preparation process of seam line 1:
S1: Preparation of suture raw materials: adopting high-purity polypropylene particles or other suitable polymer materials (such as polytetrafluoroethylene, polyester fibers, etc.) to ensure the biocompatibility and mechanical properties of the materials meet medical standards; drying polypropylene particles to remove moisture from the particles and preventing the occurrence of bubbles or uneven melting during heating, wherein the drying temperature is controlled between 70° C. and 80° C. for 4 to 6 hours.
S2:Adding the dried polypropylene particles to a melting equipment and heat them to 180° C. to 220° C. to completely melt the particles into a uniform liquid state. During this process, it is necessary to maintain a constant temperature and pressure to ensure uniform melting and plasticization of the material.
S3:Transport the melted polypropylene to a forming mold by using a screw extruder. The mold needs to be precisely designed to match the target structure of the suture thread, including the shape and size of the drainage channel and side groove. The extrusion speed of the extruder is controlled at 10 to 20 meters per minute, adjusted according to the material properties and the size of the suture to ensure stable and continuous shape of the extruded suture.
S4:The extruded suture immediately enters the stretching stage, and the suture is stretched bidirectionally or unidirectionally through a stretching device set at the outlet of the extruder. The stretching ratio is usually in the range of 2 to 5 times, and the mechanical properties and surface smoothness of the suture are optimized by adjusting the stretching rate and temperature. The temperature of the material during stretching is controlled between 90° C. and 110° C. to prevent material embrittlement or fracture caused by excessive stretching.
S5:Establishing the first drainage channel: using in mold molding or cutting method. In mold forming method is a method that a shape of drainage grooves is set in the mold, so that drainage grooves are formed during the extrusion process through precision channels in the mold. The cutting method is a method using laser to cut a drainage groove on extruded suture thread.
S6:putting the stretched suture to enter a cooling and shaping stage, let the stretched suture rapidly cooled to room temperature (about 20° C. to 25° C.) through water bath cooling or air cooling. The cooling rate should be uniform to avoid warping or surface defects of the suture caused by uneven cooling. Water bath cooling usually uses flowing water to maintain stable cooling temperature.
S7:Surface treatment is carried out on the cooled and fixed suture thread, and anti-infective, anticoagulant or lubricating coatings is applied on the suture thread according to the requirements. Dip coating or spray coating technology can be used to ensure the uniformity and effective adhesion of the coating.

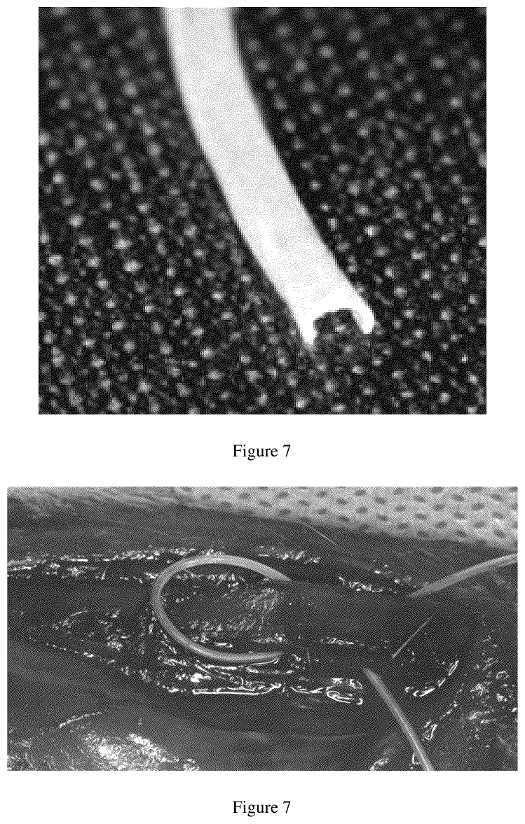

By above preparation process, a suture thread as shown in FIG. 6-7 of the instruction manual is obtained. The middle of the suture thread is provided with a drainage channel, and a side groove runs along the longitudinal axis of suture thread 1.

The size of suture thread 1 can be adjusted according to different surgical areas, and the suture thread 1 in this embodiment can be applied for tibial anterior muscle suturing, etc.

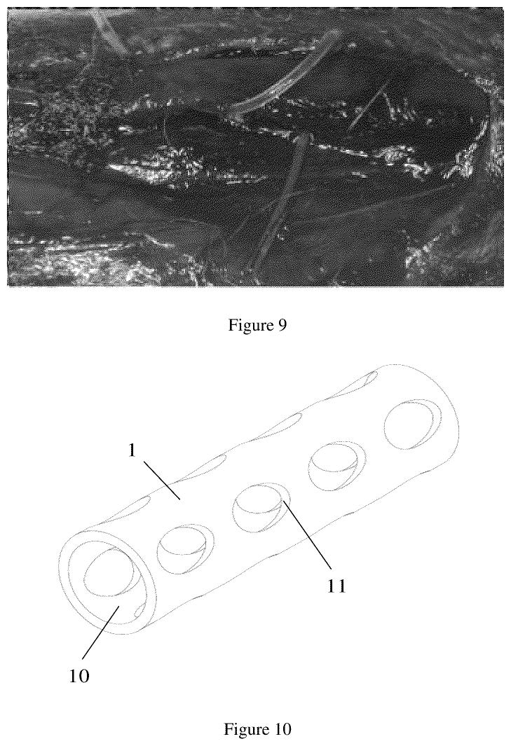

As shown in FIG. 8-9, FIG. 8 shows a suturing process with suture thread without drainage channel, that sutures the anterior tibialis muscle of a New Zealand white rabbit. FIG. 9 shows a suturing process with suture thread of the present invention this embodiment, that sutures the anterior tibialis muscle of a New Zealand white rabbit. Comparing FIGS. 8 and 9, it can be seen that after suturing the surgical area with the suture without drainage channel, all the blood seeping out of the surgical area accumulates in the surgical area, causing local swelling and inflammatory reactions. However, after suturing with the single strand suture with internal drainage channel, it can be seen that the blood seeping out of the surgical area can flow out along the drainage channel, and the blood stasis in the surgical area is greatly drained outside the surgical area. This provides powerful conditions for wound healing after suturing, greatly avoiding the risk of local swelling and inflammation. The inflammatory response caused by polarization of blood clots.

As shown in FIG. 10, it is a variant of the suture thread. It includes multiple holes 11 arranged along the side of suture thread 1, which are connected to the drainage channel 10 inside suture thread 1.

The diameter of each hole 11 is less than half of the diameter of the suture thread, and the specific size can be adjusted according to the overall diameter of the suture thread and its using scenario. Smaller pore sizes are suitable for low osmolarity environments, while larger pore sizes are suitable for high osmolarity or drainage needs of large wounds. In one implementation, the holes 11 are symmetrically arranged along the longitudinal direction of the suture thread, and two adjacent columns of holes 11 in the longitudinal direction are arranged in a staggered manner. Such arrangement ensures that each area receives effective drainage support without affecting the overall strength of the suture.

Multiple holes 11 provide a path for multi-point drainage, suitable for surgical scenarios with uneven exudate, such as deep tissue or multi site exudate treatment. Multi point drainage can more flexibly adapt to the distribution of exudate in the surgical area, effectively reducing fluid accumulation and improving overall drainage efficiency. The design of holes 11 can also reduce the compression of surrounding tissues by sutures during surgery, alleviate pain and discomfort. Suitable for surgeries that require dispersed drainage.

### One Variant of the Suture Thread

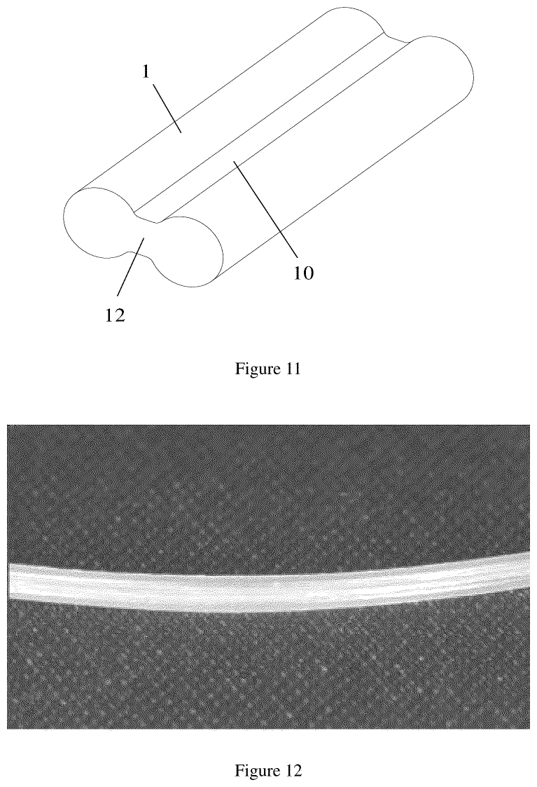

As shown in FIG. 11, it provides a variant of suture thread. The structure of suture thread 1 has a multi-strand thread body, and each strand thread body is connected to each other through a connecting part 12. The connecting part 12 is arranged along the path of suture thread 1, and the connecting part 12 is concave inward relative to the surface of the thread body connected to it. The concave serves as a drainage channel 10 for draining exudate during surgery. When in use, suture thread 1 follows the suture needle into the interior of the tissue. Due to the fact that suture thread 1 is a multi-stranded thread body, they can slightly stretch the needle path of the tissue. This design ensures that fluids within the organization can be expelled through the depression, reducing the risk of postoperative infection.

This type of suture can be composed of 2 to 6 strands of thread. For example, in one design, the suture is composed of two parallel solid thread bodies connected by a connecting portion 12. This type of suture appears dumbbell shaped in cross-section when not under stress. The connecting part 12 can adopt different shapes, for example, the upper and lower end faces of the connecting part 12 can be flat or curved.

It should be noted that the preparation process of the suture thread 1 (melting and plasticizing of raw materials, extrusion molding, stretching, cooling and shaping) is the same as that of the suture thread 1 above. By replacing the molding mold inside the extruder, the preparation of the suture 1 in this variant can be achieved. Thus, the suture thread as shown in FIG. 12 can be obtained.

The size of suture thread 1 can be adjusted according to different surgical areas. The suture thread 1 in this variant can be used for suturing mucous membranes, skin, and organs based on thickness and tensile strength.

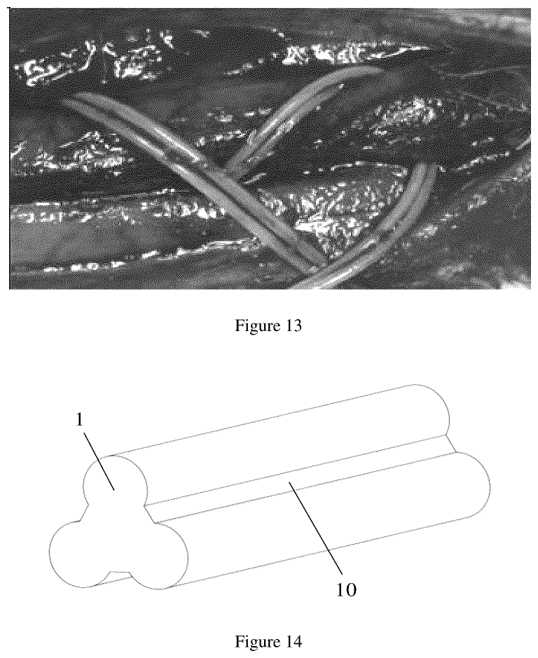

As shown in FIGS. 8 and 13, FIG. 8 shows a process that the suture structure without drainage channel is used for suturing the anterior tibialis muscle of a New Zealand white rabbit. FIG. 13 shows a process that the suture structure of this variant is used for suturing the anterior tibialis muscle of a New Zealand white rabbit. Comparing FIGS. 8 and 13, it can be seen that after using the suture without drainage channel, all the blood leaked from the surgical area accumulates in the surgical area, causing local swelling and inflammatory reactions. However, after using the double stranded suture with drainage channel on the surface of the suture body, it can be seen that the blood leaked from the surgical area can flow out along the drainage channels on both sides, and the accumulated blood in the surgical area is greatly drained outside the surgical area. This provides a basis for wound healing after suturing. Under favorable conditions, the inflammatory response caused by blood clot polarization was greatly avoided.

### Another Variant of the Suture Thread

As shown in FIG. 14, it is a variant of Example 2, and the suture thread 1 of this embodiment is formed by fusing three solid thread bodies. The cross-sectional shape of this suture thread is like a triangle with a dome. Similar to Example 2, the thread bodies are also connected to each other through a connecting part 12, and the connecting part 12 is concave inward relative to the surface of the thread body connected to it, forming three drainage channels 10. When the suture thread 1 follows the suture needle into the tissue, it uses the outer edges of the three thread bodies to open the needle passage inside the tissue, ensuring that the liquid in the needle passage can be smoothly discharged through the three drainage channels 10.

It should be noted that the preparation process of the suture 1 with the structure of this embodiment (melting and plasticizing of raw materials, extrusion molding, stretching, cooling and shaping) is the same as that of the suture 1 in embodiment 1. By replacing the molding mold inside the extruder, the preparation of the suture 1 in this embodiment can be achieved.

The size of suture 1 in this embodiment can be adjusted according to different surgical requirements. This type of suture design can be applied in various surgeries based on its thickness and tensile strength, such as suturing mucous membranes, skin, and internal organs. It can not only suture wounds, but also effectively drain exudate during surgery, helping wounds heal faster.

### Another Variant of the Suture Thread

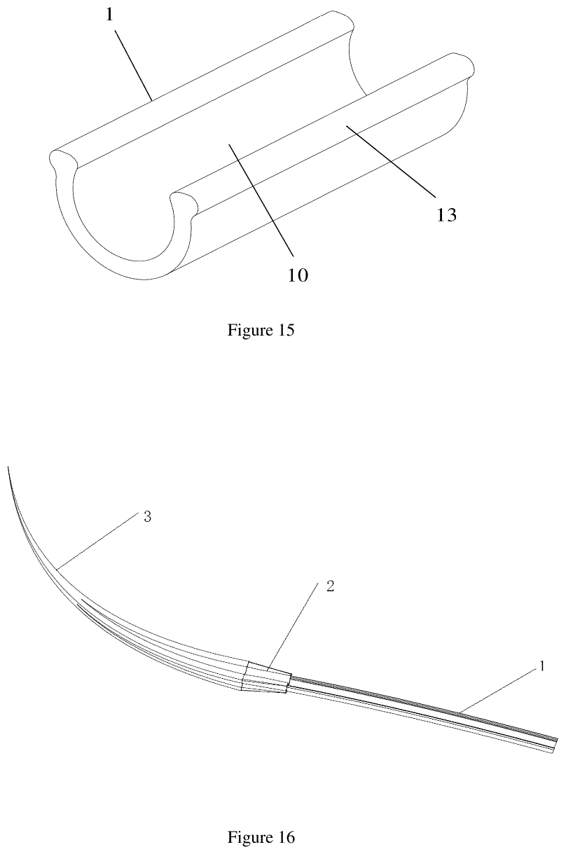

As shown in FIG. 15, it is another variation of Example 1. In this embodiment, the suture thread 1 has the same main structure as that of Example 1, both of which are designed as single stranded thread bodies. There is an opening on upper part of the suture body, and a channel in the middle of the suture body that runs along the length direction of the suture, used for draining exudate during surgery.

On both sides of the opening of the suture body, there are suture side wings 13 extending outward. Each side wing extends along the longitudinal length of the suture body from one end to the other, forming a continuous structure. The lateral wings of the suture can slightly expand the surrounding soft tissue through their external expansion form, forming a more spacious drainage path. This stretching effect reduces the direct compression of the suture on the surrounding tissue and reduces the direct contact area between the outer surface of the suture and the tissue.

The bottom of suture flank 13 is inwardly recessed at the junction with the outer surface of the suture body. Although this recess does not directly connect to the opening, it can serve as a part of the surface drainage path, creating a small negative pressure environment that guides some exudate to flow along the outer surface of suture 1, assisting in promoting the outward discharge of tissue fluid. This design can reduce fluid retention and lower the risk of infection in a sterile environment.

The using method of the surgical suturing instrument in this embodiment is as below:
Step 1: Hold the needle with the right hand at the back ⅓ part of the needle body, and use tweezers with the left hand to keep the tissue to be sutured.
Step 2: Align the needle tip with the point for needle inserting and insert it perpendicular to the tissue; rotate the needle holder in place; insert the needle along the curvature of the suture needle and exit at the corresponding symmetrical point on the opposite side.

The type of suture needle 3 can be selected according to the exposed space and environment of different surgical sites. When the surgical site is the skin, eyes, blood vessels, nerves, etc., a needle type of ⅜ arc (i.e., an arc which is a part of ⅜ of a whole cycle) can be selected for suture operation. When the surgical site is in the gastrointestinal tract, muscles, or other areas, a needle type of ½ arc can be selected for suturing operation. When the surgical site is in the pelvic cavity, urogenital tract, or other areas, a needle type of ⅝ arc can be selected for suturing operation. When the surgical site is the eye, oral cavity, or other areas, a composite curved needle can be selected for suturing operation.
Step 3: After the suture needle 3 passes through the tissue, go on guiding the transition part 2 (as shown in FIG. 13) through the tissue. As the cross-section of the transition part 2 gradually expands from the end of the suture needle 3 to the end of the suture thread 1, the transition part 2 extending along the longitudinal axis of the suture needle 3 will pass through the pierced tissue during the pulling and suturing process and support the larger opening at the pierced tissue.
Step 4: Continue to pull the suture needle 3 that passes through the tissue, so that the suture 1 following the transition part 2 also passes through the punctured tissue and stays in the body. The drainage channel 10 on suture 1 guides the blood and tissue fluid in the surgical area to flow out of the body along suture thread 1. Knot and cut suture thread 1 to complete the suture.

The above only describes embodiments of the present invention, and the specific structures and characteristics commonly known in the scheme are not described in detail here. For those skilled in the art, it is apparent that the present invention is not limited to the details of the exemplary embodiments described above, and can be implemented in other specific forms without departing from the spirit or essential features of the present invention. Therefore, from any point of view, the embodiments should be regarded as exemplary, but not limiting, and the scope of the present invention is limited by the appended claims rather than the above description. Therefore, it is intended to encompass all variations falling within the meaning and scope of the equivalent elements of the claims within the scope of the present invention. Any reference numerals in the claims should not be regarded as limiting the claims involved.

1. A surgical suturing instrument for reducing fluid accumulation around suture site, wherein the surgical suturing instrument comprises a suture needle and a suture thread, connected with each other; at least one guide groove is provided on the suture needle, wherein the suture thread comprises a suture body;
a drainage channel is provided in the middle or at a side of the suture body, which extends along its length direction;
the drainage channel is connected to a tissue needle channel which is caused by a needle that passing through the tissue during suturing process, and the drainage channel is used to drain fluid that seeped out of the tissue needle channel and suture area, wherein the suture thread includes a single strand thread body or a multi-strand thread body;
wherein the single stranded suture body is provided with an opening on its side that is connected to the drainage channel and the tissue needle channel, which opening is used to drain the liquid in the tissue needle channel and the suture area;
the multi-strand thread body is connected to each other through a connecting part, and the connecting part is set along the path of the suture thread; wherein the connecting part is concave inward relative to the surface of respective wire body, and the concave is used as a drainage channel; the number of multi-strand thread bodies is 2-6.

2. The surgical suturing instrument according to claim 1, wherein the openings are multiple holes distributed along the outer surface of the suture thread.

3. The surgical suturing instrument according to claim 1, wherein the drainage channel extends along the longitudinal axis direction of the suture thread.

4. The surgical suturing instrument according to claim 1, wherein at edge of the opening of the suture thread of the single strand suture body, there is a suture side wing extending outward, and the suture side wing extends from one end to the other along the longitudinal axis of the suture body.

5. The surgical suturing instrument according to claim 1, wherein a cross-section of the drainage channel of the single stranded suture body has a shape of one of below shapes: crescent shape, semi-circular shape, square shape, and elliptical shape.

6. The surgical suturing instrument according to claim 1, wherein the material of the suture comprises non degradable materials and/or biodegradable materials; the non-degradable materials include one or more of nylon, polytetrafluoroethylene, polyester fiber, stainless steel, nickel chromium alloy, titanium, and titanium alloys; biodegradable materials include one or more of polyacetic acid, polylactic acid hydroxyacetic acid copolymer, polyhydroxyacetic acid ethyl ester, magnesium and magnesium alloys, iron and iron alloys, zinc and zinc alloys.

7. The surgical suturing instrument according to claim 1, wherein the inner and outer surfaces of the suture thread are coated with a hydrophobic coating containing anti-infective drugs and anticoagulant drugs; Or coating with hydrophilic coatings containing anti-infective and anticoagulant drugs.

8. The surgical suturing instrument according to claim 7, wherein the guide groove is longitudinally arranged on the surface of the needle body of the suture needle, and the guide groove starts from the part of the needle body near the needle tip, smoothly extends along the curved surface of the suture needle, to the needle tail.
